(19) United States
(12) Patent Application Publication
(10) Pub. No.: US 2024/0398718 A1
(43) Pub. Date: Dec. 5, 2024

(54) ECTOSOME-BIODEGRADABLE POLYMER NANOPARTICLE COMPLEX WITH ENHANCED TARGETING TO LESIONS AND ITS METHOD OF PREPARATION

(71) Applicant: GACHON UNIVERSITY OF INDUSTRY-ACADEMIC COOPERATION FOUNDATION, Seongnam-si (KR)

(72) Inventors: Dongwoo Khang, Seongnam-si (KR); Jun-Young Park, Seongnam-si (KR); Young-Gyu Jeong, Seongnam-si (KR)

(73) Assignee: GACHON UNIVERSITY OF INDUSTRY-ACADEMIC COOPERATION FOUNDATION, Seongnam-si (KR)

(21) Appl. No.: 18/804,871

(22) Filed: Aug. 14, 2024

Related U.S. Application Data
(63) Continuation-in-part of application No. PCT/KR2023/001914, filed on Feb. 9, 2023.

(30) Foreign Application Priority Data
Feb. 14, 2022 (KR) 10-2022-0019192
Feb. 14, 2022 (KR) 10-2022-0019193

(51) Int. Cl. A61K 9/50; A61K 9/51

(52) U.S. Cl. CPC A61K 9/5068; A61K 9/5153; A61K 9/5161; A61K 9/5169

(57) ABSTRACT

The present invention relates to novel ectosome-biodegradable polymer nanoparticle complexes, and more specifically, to biodegradable polymer nanoparticle complexes coated with stem cell-derived ectosome membranes and methods of preparing the ectosome-biodegradable polymer nanoparticle complexes with enhanced targeting ability to lesions.

### CROSS-REFERENCE TO RELATED APPLICATION

This application is a continuation-in-part of PCT/KR2023/001914 filed Feb. 9, 2023, which claims priority to Korean Patent Application No. 10-2022-0019192 filed Feb. 14, 2022 and Korean Patent Application No. 10-2022-0019193 filed Feb. 14, 2022, the contents of each of which are incorporated by reference in their entirety.

### FIELD

The present invention relates to a novel ectosome-biodegradable polymer nanoparticle complex and its method of preparation, and more particularly to a novel ectosome-biodegradable polymer nanoparticle complex with enhanced targeting to tumor and inflammatory sites and its method of preparation.

### BACKGROUND

Ectosomes are vesicles of various sizes (0.1 to 1 μm in diameter) that sprout directly from the plasma membrane and are secreted into the extracellular space. Unlike living cells, ectosomes have phosphatidylserine, a type of phospholipid, on their surface. Despite the fact that ectosomes are produced and secreted by direct budding of the cell membrane, their internal cargo is known to be different from of the parent cells, but to vary depending on the state of the cells (Cocucci and Meldolesi, Curr. Biol. 21(23): R940-R940, 2011). Although ectosomes differ from exosomes, which are secreted extracellularly from endosomes via the multivesicular body (MVB) pathway, in terms of way of formation and containing substances, they are known to be similar to exosomes in their interactions in the extracellular space and their interactions with target cells. However, while exosomes have attracted much attention in terms of their function in disease treatment and diagnosis, ectosomes have been relatively less studied.

Relevant prior art includes U.S. Pat. No. 2,162,727, which discloses a cell therapy composition for cancer treatment comprising Natural Killer Cells and Extracellular Vesicles derived from the cells.

### SUMMARY OF THE DISCLOSURE

However, in the case of the above prior art, it is unclear whether the extracellular vesicles are ectosomes or exosomes, and they alone have little anti-cancer therapeutic effect.

The present invention is intended to address the above and other problems, and aims to provide a novel ectosome-derived drug delivery carrier with enhanced targeting to tumor and inflammatory sites, and its method of preparation. However, these tasks are exemplary and the scope of the present invention is not limited thereto.

In one aspect of the present invention, there is provided a biodegradable polymer nanoparticle coated with membrane of a stem cell-derived ectosome.

In another aspect of the present invention, there is provided a drug delivery carrier comprising the biodegradable polymer nanoparticle coated with membrane of a stem cell-derived ectosome.

In another aspect of the present invention, there is provided a pharmaceutical composition comprising the drug delivery carrier and an active drug loaded into the drug delivery carrier.

In another aspect of the present invention, there is provided a pharmaceutical composition for the treatment of cancer, comprising a nanoparticle-drug complex comprising a biodegradable polymer nanoparticle coated with a membrane of a stem cell-derived ectosome and an anticancer agent loaded to the nanoparticle as an active ingredient.

In another aspect of the present invention, there is provided a method of treating cancer in a subject suffering from cancer, comprising administering the pharmaceutical composition to the subject.

In another aspect of the present invention, there is provided a pharmaceutical composition for the treatment of inflammation, comprising a nanoparticle-drug complex comprising a biodegradable polymer nanoparticle coated with a membrane of a stem cell-derived ectosome and an anti-inflammatory agent loaded to the nanoparticle as an active ingredient.

In another aspect of the present invention, there is provided a method of treating inflammation in a subject suffering from an inflammation, comprising administering the pharmaceutical composition to the subject.

In another aspect of the present invention, there is provided a method of preparing an ectosome-biodegradable polymer nanoparticle complex with enhanced targeting to lesions, comprising preparing an educated stem cell by educating a stem cell with lesion-derived cells; isolating an ectosome from the educated stem cell; and preparing a biodegradable polymer nanoparticle coated with membrane of the ectosome, by mixing the ectosome or an membrane of the ectosome generated by degradation of the ectosome with a biodegradable polymer nanoparticle.

Because the stem cell-derived ectosome-biodegradable polymer nanoparticle complex of the present invention prepared as described above has enhanced targeting to target disease sites such as tumors or inflammatory sites, it can be used as a highly efficient anticancer and anti-inflammatory therapeutic when loaded with anticancer and anti-inflammatory drugs, respectively. Additionally, the method of preparing the complex can be utilized to develop drug delivery carriers with enhanced targeting to lesion sites. However, the scope of the present invention is not limited by these effects.

### BRIEF DESCRIPTION OF THE DRAWINGS

FIG. 1 is a schematic illustration of the preparation of ectosome-biodegradable polymer (PLGA), which is a biodegradable polymer (PLGA) coated with ectosomes (nanovesicles) derived from adipose-derived mesenchymal stem cells targeting pancreatic cancer or brain tumor, educated according to one embodiment of the present invention.

FIG. 2 is a transmission electron microscopy (TEM) image showing that ectosomes are well coated on PLGA, after the preparation of ectosome-biodegradable polymer (PLGA) coated with ectosomes (nanovesicles) isolated from adipose-derived mesenchymal stem cells targeting tumors, which were educated with tumor cells according to one embodiment of the present invention.

FIG. 3 shows a series of graphs showing the results of analyzing hydrodynamic sizes (a) and electrical potentials (b) of PLGA, PLGA-DID, ectosomes derived from AD-MSC, PLGA-DID coated with ectosomes derived from AD-MSC, ectosomes derived from AD-MSC educated with brain tumor cells (U87MG), and PLGA-DID coated with ectosomes derived from AD-MSC educated with brain tumor cells.

FIG. 4 is a graph showing the results of analyzing fluorescence intensities of PLGA-DID at an excitation wavelength of 646 nm and an emission wavelength of 661 nm, using Victor 1420 Multilabel Counter (Perkin-Elmer, Waltham, MA, USA).

FIG. 5 is a series of fluorescence microscopic images that visualizes the uptake of PLGA (PLGA-DIO) stained with DiO which is a fluophore in the GFP wavelength range, PLGA coated with ectosomes isolated from adipose-derived mesenchymal stem cells (AD-MSC) stained with DiO, and PLGA coated with ectosomes extracted from educated AD-MSC stained with the fluophore DID, in order to assess the ability of ectosome-PLGA to migrate into pancreatic cancer cells (PANC-1) and brain tumor cells (U87MG).

FIG. 6a is a photograph showing the results of whole-body biofluorescence imaging of the in vivo distribution after injection of the polymer (PLGA) coated with ectosomes (nanovesicles) isolated from tumor-targeting adipose-derived stem cells educated with tumor cells according to one embodiment of the present invention, into a pancreatic cell (PANC-1) xenograft tumor model animal.

FIG. 6b is a series of biofluorescence images depicting the distribution of cancer cells (top) and ectosome-PLGA (bottom) in tumor tissue harvested from the above experimental model.

FIG. 7 shows a series of graphs showing the hydrodynamic sizes (a) and electrical potentials (b) of PLGA-DOX coated with ectosomes isolated from AD-MSC educated with PLGA, PLGA-DOX, and pancreatic cancer cells (PANC-1).

FIG. 8a is an image showing the results of whole-body biofluorescence imaging of the in vivo distribution after injection of the polymer (PLGA) coated with ectosomes (nanovesicles) isolated from tumor-targeting adipose-derived stem cells educated with tumor cells according to one embodiment of the present invention, into brain cancer cell (U87MG) xenograft tumor model animals.

FIG. 8b is a series of biofluorescence images depicting the distribution of cancer cells (top) and ectosome-PLGA (bottom) in tumor tissue excised from the above experimental models.

FIG. 9 is a series of whole-body biofluorescence images showing the in vivo distribution after injection of the polymer (PLGA) coated with ectosomes (nanovesicles) isolated from educated adipose-derived stem cells targeting tumors, into pancreatic cell (PANC-1) and brain cell (U87MG) xenograft tumor model animals, for assessment of the tumor-targeting ability and anti-cancer effect of ectosome-PLGA-DOX complexes according to one embodiment of the present invention.

FIG. 10a is a schematic diagram illustrating experimental methods for evaluating the ability of ectosome-PLGA-nanoparticle complex derived from educated stem cells to target arthritic cells, according to one embodiment of the present invention.

FIG. 10b is a series of whole-body biofluorescence images for assessment of the targeting ability of ectosome-PLGA nanoparticle complex derived from stem cells educated according to one embodiment of the present invention (left), a series of confocal microscopic images for assessment of the uptake in inflammatory synoviocytes (FLSs) and inflammatory macrophages (J774) (upper right), and a graph showing the quantification of fluorescence intensities in the above confocal microscopic images (lower right).

### DETAILED DESCRIPTION OF THE DISCLOSURE

Definitions of terms:

As used herein, the term “ectosome” refers to a phospholipid membrane-structured nanovesicle with a diameter of 100 to 500 nm that is generated by budding of plasma membrane, as opposed to “exosome” among extracellular vesicles.

As used herein, the term “biodegradable polymer” refers to a physiologically harmless polymer that is subject to degradation by enzymatic actions in vivo. Biodegradable polymers include natural polymers such as starch, chitin, cellulose, polyalginate, and collagen, and man-made polymers such as PLGA {poly(lactic-co-glycolic) acid}, PGA {poly(glycolic acid)}, PLA {poly(lactic acid)}, PCL {poly(caprolactone)}, and PHA (polyhydroxyalkanoate).

As used herein, the term “drug delivery carrier” refers to a substance used to deliver a drug to the sites in need thereof and retain it for an appropriate amount of time, and the method of effectively delivering a drug to a site using a drug delivery carrier is referred to as a drug delivery system.

A detailed description of the disclosure:

In one aspect of the present invention, there is provided a biodegradable polymer nanoparticle coated with a membrane of a stem cell-derived ectosome.

In the nanoparticle, the stem cell may be an embryonic stem cell, a mesenchymal stem cell, or an induced pluripotent stem cell, and the mesenchymal stem cell may be a bone marrow-derived stem cell, a cord blood-derived stem cell, an adipose-derived stem cell, a dental pulp-derived stem cell, or a peripheral blood-derived stem cell.

In the nanoparticle, the biodegradable polymer may be a natural biodegradable polymer or an artificial biodegradable polymer, wherein the natural biodegradable polymer may be starch, chitin, cellulose, polyalginate, or collagen, and the artificial biodegradable polymer may be PLGA {poly(lactic-co-glycolic) acid}, PGA {poly(glycolic acid)}, PLA {poly(lactic acid)}, PCL {poly(caprolactone)}, or PHA (polyhydroxyalkanoate).

The nanoparticle may have a size of 100 to 350 nm in diameter.

In another aspect of the present invention, there is provided a drug delivery carrier comprising the nanoparticle.

In another aspect of the present invention, there is provided a pharmaceutical composition comprising the drug delivery carrier and an active drug loaded into the drug delivery carrier.

In the pharmaceutical composition, the active drug may be an anti-cancer agent or an anti-inflammatory agent.

Here are examples of the anti-cancer agent:
(I) asparaginase;
(II) methotrexate;
(III) pyrimidine analogs
Examples: 5-fluorouracil, gemcitabine, and arabinosylcytosine;
(iv) hydroxy urea;
(v) purine analogs
Mercaptopurine and thioguanine;
(vi) alkylating agents
Nitrogen mustard and cyclosporamide;
(vii) anthracyclines
Anthracycline, doxorubicin, daunorubicin, idarubicin, and actinomycin D;
(viii) mitotic inhibitors
Vincristine and taxol;
(ix) Antiangiogenic agents

Antibodies specific to VEGF, combretastatin A4, fumagillin, herbimycin A, 2-methoxyestradiol, OGT 2115, TNP 470, tranilast, XRP44X, thalidomide, endostatin, salmosin, angiostatin, or plasminogen, or the kringle domain of apolipoprotein;
(x) intercalating agents
carboplatin and cisplatin;
(xi) radionuclides
¹⁸F, 90Y, ¹⁸⁸Re, ³²P ⁸⁹Sr, ¹⁶⁵Dy, ¹⁸⁶Re, ¹⁹⁸Au, ¹⁵³Sm, ¹³¹I, ¹⁶⁹Er, ¹²⁵I, ⁹⁹Tc, and ¹⁶⁶Ho, etc.

In the pharmaceutical composition, the anti-inflammatory agent may be a glucocorticoid or a non-steroidal anti-inflammatory agent.

In the pharmaceutical composition, the glucocorticoid may be hydrocortisone, hydrocortisone acetate, cortisone, cortisone acetate, tixocortol pivalate, hydrocortisone-17-valerate, halometasone, alclometasone dipropionate, and betamethasone valerate, betamethasone dipropionate, prednicarbate, clobetasone-17-butyrate, and clobetasol-17-propionate, fluocortolone caproate, fluocortolone pivalate, fluprednidene acetate, prednisone, and prednisolone, methylprednisolone, dexamethasone, dexamethasone sodium phosphate, betamethasone, bethamethasone sodium phosphate, fluocortolone, triamcinolone, triamcinolone acetonide, mometasone, amcinonide, desonide, and fluocinonide, fluocinolone acetonide, hacinonide, beclomethasone, fludrocortisone acetate, hydrocortisone-17-butyrate, hydrocortisone aceponate, hydrocortisone-17-buteprate, ciclesonide, or prednicarbate.

In the pharmaceutical composition, the non-steroidal anti-inflammatory agent may be a cyclooxygenase (COX) inhibitor. Further, the cyclooxygenase inhibitor may be a non-selective COX-1/COX-2 inhibitor, a selective COX-1 inhibitor, or a selective COX-2 inhibitor, wherein the selective COX-2 inhibitor may be apricoxib, celecoxib, rofecoxib, parecoxib, lumiracoxib, etoricoxib, or firocoxib.

In another aspect of the present invention, there is provided a pharmaceutical composition for the treatment of cancer, comprising a nanoparticle-drug complex comprising a biodegradable polymer nanoparticle coated with a membrane of a stem cell-derived ectosome and an anticancer agent loaded to the nanoparticle as an active ingredient.

In the pharmaceutical composition, the stem cell may be a stem cell educated with target cancer cells, and the anticancer agent may be as described above.

Furthermore, the manner in which the anticancer agent is loaded into the biodegradable polymer nanoparticle may be by covalent or non-covalent bond to the surface or interior of the nanoparticle, or by inclusion inside the nanoparticle having a core-shell structure. In the case of loading by covalent bonding, the anticancer agent may be covalently bound to the end or side chains of the biodegradable polymer. Optionally, loading may also be accomplished in such a way that the anticancer agent is covalently or non-covalently bound to and presented on the surface of the externally coated ectosome membrane.

In another aspect of the present invention, there is provided a method of treating cancer in a subject suffering from cancer, comprising administering the pharmaceutical composition to the subject.

In another aspect of the present invention, there is provided a pharmaceutical composition for the treatment of inflammation comprising a nanoparticle-drug complex comprising a biodegradable polymer nanoparticle coated with a membrane of a stem cell-derived ectosome and an anti-inflammatory loaded to the nanoparticle as an active ingredient.

In the pharmaceutical composition, the stem cell may be an educated stem cell educated with an inflammatory cell obtained from the target inflamed site, and the anti-inflammatory agent may be as described above.

Furthermore, the manner in which the anti-inflammatory agent is loaded into the biodegradable polymer nanoparticle may be by covalent or non-covalent bond to the surface or interior of the nanoparticle, or by inclusion inside the nanoparticle having a core-shell structure. In the case of loading by covalent bond, the anti-inflammatory agent may be covalently bound to the end or side chains of the biodegradable polymer. Optionally, loading may also be accomplished in such a way that the anti-inflammatory agent is covalently or non-covalently bound to and presented on the surface of the externally coated ectosome membrane.

In another aspect of the present invention, there is provided a method of treating inflammation in a subject suffering from inflammation, comprising administering the pharmaceutical composition to the subject.

The pharmaceutical compositions of the present invention can be varied depending on the type of patient, the site of application, the number of treatments, the time of treatment, the formulation, the patient's condition, the type of adjuvant, etc. The dosage is not particularly limited, but may be from 0.01 μg/kg/day to 10 mg/kg/day. The daily dose may be administered once daily, or two to three times daily at reasonable intervals, or intermittently over several days.

The pharmaceutical compositions of the present invention can be administered orally or parenterally, preferably parenterally, for example by intravenous injection, subcutaneous injection, intracerebroventricular injection, intracerebrospinal fluid injection, intramuscular injection, and intraperitoneal injection.

The pharmaceutical compositions of the present invention may further comprise suitable carriers, excipients and diluents that are commonly used in the preparation of pharmaceutical compositions. In addition, solid or liquid formulation additives may be used in the preparation of the pharmaceutical compositions. The formulation additives may be organic or inorganic. Excipients include, for example, lactose, sucrose, white sugar, glucose, cornstarch, starch, talc, sorbitol, crystalline cellulose, dextrin, kaolin, calcium carbonate, and silicon dioxide. Binding agents include, for example, polyvinyl alcohol, polyvinyl ether, ethylcellulose, methylcellulose, gum arabic, tragacanth, gelatin, shellac, hydroxypropylcellulose, hydroxypropylmethylcellulose, calcium citrate, dextrin, and pectin. Glossing agents include, for example, magnesium stearate, talc, polyethylene glycol, silica, and hydrogenated vegetable oils. Colorants can be used as long as they are approved for addition to pharmaceutical products. The tablets and granules may be sugar coated, gelatinized, or otherwise coated as appropriate. In addition, preservatives, antioxidants, and the like may be added as needed. In addition, when the pharmaceutical composition is a therapeutic agent, it may further include one or more of the following selected from fillers, anticoagulants, lubricants, wetting agents, flavors, emulsifiers, or preservatives. The formulations of the pharmaceutical compositions of the present invention may be in any desirable form depending on the method of use, and are particularly preferably formulated by employing methods known in the art to provide a rapid, sustained, or delayed release of the active ingredients after administration to the mammal. Examples of specific formulations include plasters, granules, lotions, liniments, lemonades, powders, syrups, liquids and solutions, aerosols, extracts, elixirs, fluidextracts, emulsions, suspensions, decoctions, infusions, tablets, suppositories, injections, spirits, cataplasmas, capsules, troches, tinctures, pastes, pills, and soft or hard gelatin capsules.

The pharmaceutical compositions of the present invention may further contain additional ingredients commonly used, such as carriers, and conventional auxiliaries including stabilizers, solubilizers and flavorings.

The pharmaceutical compositions or intra-articular injections may comprise a variety of carriers suitable for direct injection into the affected area.

Pharmaceutically acceptable carriers included in the pharmaceutical compositions of the present invention are those customarily utilized in pharmaceutical preparations, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, but are not limited by these examples. In addition to the above ingredients, the pharmaceutical compositions of the present invention may further comprise lubricants, wetting agents, sweeteners, flavoring agents, emulsifiers, suspending agents, preservatives, and the like. Suitable pharmaceutically acceptable carriers and formulations are described in detail in Remington's Pharmaceutical Sciences (19^{th} Ed., 1995).

In another aspect of the present invention, there is provided a method of preparing an ectosome-biodegradable polymer nanoparticle complex with enhanced targeting ability to lesions, comprising preparing an educated stem cell by educating a stem cell with a lesion-derived cell; isolating ectosomes from the educated stem cell; mixing the ectosome or an membrane of ectosome generated by degradation of the ectosomes with a biodegradable polymer nanoparticle to prepare an ectosome membrane-coated biodegradable polymer nanoparticle.

In the above method, the membrane of ectosome may be produced by mechanical extrusion or sonication of the ectosomes.

Furthermore, the degradation of the ectosomes and the coating of the biodegradable polymer nanoparticles with the ectosome membrane can be performed simultaneously or sequentially. When performed simultaneously, ectosomes are first mixed with biodegradable polymer nanoparticles and then sonicated, which allows the simultaneous achievement of degradation and coating of the ectosome membrane. Conversely, ectosome membrane degradation can be first achieved by sonicating isolated ectosomes, followed by ectosome membrane coating by mixing the resulting ectosome membrane fragments with biodegradable polymer nanoparticles. Methods for coating nanoparticles with extracellular vesicles (EVs) such as ectosomes are well described in the prior art (Fathi et al., VIEW, 2(2): 20200187, 2020; Van Deun et al., Cells, 9(8): 1797, 2020).

The mechanical extrusion may be performed by passing the ectosome through a membrane for the use of extrusion with holes of a suitable size (20 to 200 nm), and the membrane of ectosome produced by the extrusion process may be mixed with biodegradable polymer nanoparticle to prepare ectosome membrane-coated biodegradable polymer nanoparticle. Besides, the ectosome membrane-coated biodegradable polymer nanoparticle may be prepared by secondary extrusion of the mixture of ectosome membrane and biodegradable polymer nanoparticle (van Deun et al., Cells, 9(8): 1797, 2020).

In the above method, the stem cell may be an embryonic stem cell, mesenchymal stem cell, or an induced pluripotent stem cell, and the mesenchymal stem cell may be bone marrow-derived stem cell, a cord blood-derived stem cell, an adipose-derived stem cell, a dental pulp-derived stem cell, or a peripheral blood-derived stem cell, and the education may be performed by culturing the stem cell in contact with the culture medium in which the lesion-derived cells are cultured.

In the above preparation method, the biodegradable polymer may be a natural biodegradable polymer or an artificial biodegradable polymer, wherein the natural biodegradable polymer may be starch, chitin, cellulose, polyalginate, or collagen, and the artificial biodegradable polymer may be PLGA {poly(lactic-co-glycolic) acid}, PGA {poly(glycolic acid)}, PLA {poly(lactic acid)}, PCL {poly(caprolactone)}, or PHA (polyhydroxyalkanoate).

In the method, the nanoparticle may have a size of 100 to 350 nm in diameter and may be loaded with an active drug, wherein the active drug may be the anticancer agent or the anti-inflammatory agent.

In the method, the anticancer agent may be one among described above.

In the method, the anti-inflammatory agent may be a glucocorticoid or a non-steroidal anti-inflammatory agent.

In the method, the glucocorticoid may be hydrocortisone, hydrocortisone acetate, cortisone, cortisone acetate, tixocortol pivalate, hydrocortisone-17-valerate, halometasone, alclometasone dipropionate, and betamethasone valerate, betamethasone dipropionate, prednicarbate, clobetasone-17-butyrate, and clobetasol-17-propionate, fluocortolone caproate, fluocortolone pivalate, fluprednidene acetate, prednisone, and prednisolone, methylprednisolone, dexamethasone, dexamethasone sodium phosphate, betamethasone, bethamethasone sodium phosphate, fluocortolone, triamcinolone, triamcinolone acetonide, mometasone, amcinonide, desonide, and fluocinonide, fluocinolone acetonide, hacinonide, beclomethasone, fludrocortisone acetate, hydrocortisone-17-butyrate, hydrocortisone aceponate, hydrocortisone-17-buteprate, ciclesonide, or prednicarbate.

In the above method, the non-steroidal anti-inflammatory agent may be a cyclooxygenase (COX) inhibitor. Further, the cyclooxygenase inhibitor may be a nonselective COX-1/COX-2 inhibitor, a selective COX-1 inhibitor, or a selective COX-2 inhibitor, wherein the selective COX-2 inhibitor may be apricoxib, celecoxib, rofecoxib, parecoxib, lumiracoxib, etoricoxib, or firocoxib.

Furthermore, in the above preparation method, the manner in which the active drug is loaded into the biodegradable polymer nanoparticle may be by covalent or non-covalent bonding to the surface or interior of the nanoparticle, or by inclusion inside the nanoparticle of a core-shell structure. In the case of loading by covalent bonding, the active drug may be covalently bound to the end or side chains of the biodegradable polymer. Optionally, loading may also be accomplished in such a way that the active drug is covalently or non-covalently bound to and presented on the surface of the externally coated ectosome membrane.

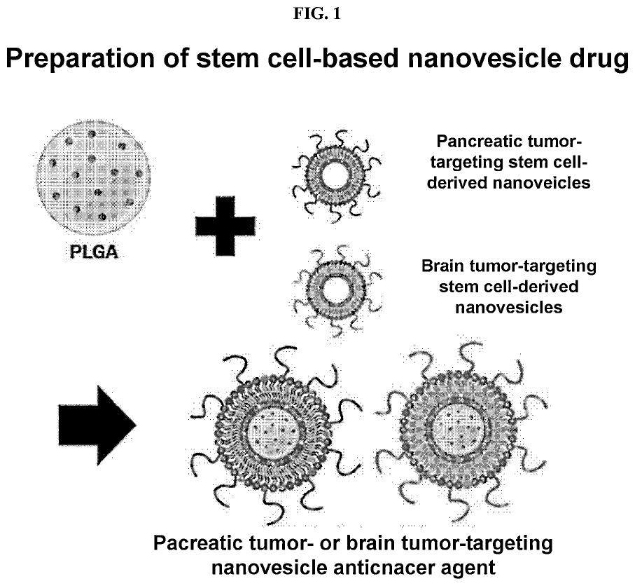

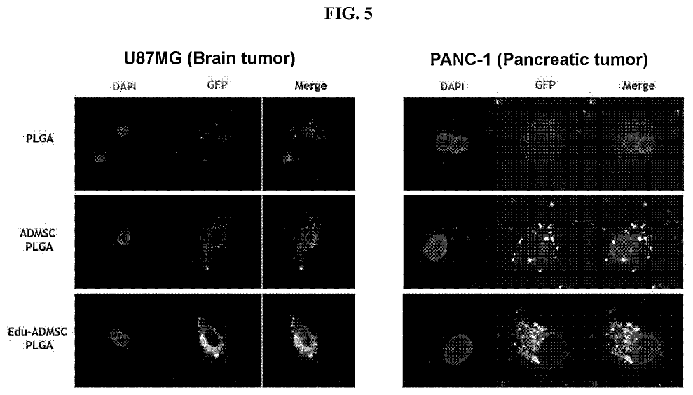

The present inventors have shown in prior studies that stem cells educated with cancer cell or inflammatory cell cultures have enhanced targeting ability to tumor tissue or inflammatory tissue. Based on this, the present inventors hypothesized that ectosomes secreted from the stem cells educated as described above would also have the ability to target tumor tissue or inflammatory tissue. To prove the above hypothesis, ectosomes were isolated from educated stem cells and mixed with the biodegradable polymer PLGA nanoparticles, to prepare ectosome-biodegradable nanoparticle complexes coated with ectosome membranes (see FIG. 1), and it was investigated whether these nanoparticle complexes target tumor tissue and inflammatory tissue effectively. The results confirmed that the ectosome-biodegradable nanoparticle complexes comprising the ectosomes isolated from the stem cells educated according to one embodiment of the present invention exhibit significantly higher tumor cell targeting ability compared to uneducated stem cell-derived ectosome-biodegradable polymer nanoparticle complexes (see FIGS. 5, 6 and 8). Furthermore, when the ectosome-biodegradable polymer nanoparticle complexes were loaded with anticancer drugs such as doxorubicin and administered to tumor model mice, tumor growth was significantly inhibited compared to the administration of anticancer drugs alone or biodegradable polymer nanoparticles loaded with anticancer drugs (see FIG. 9).

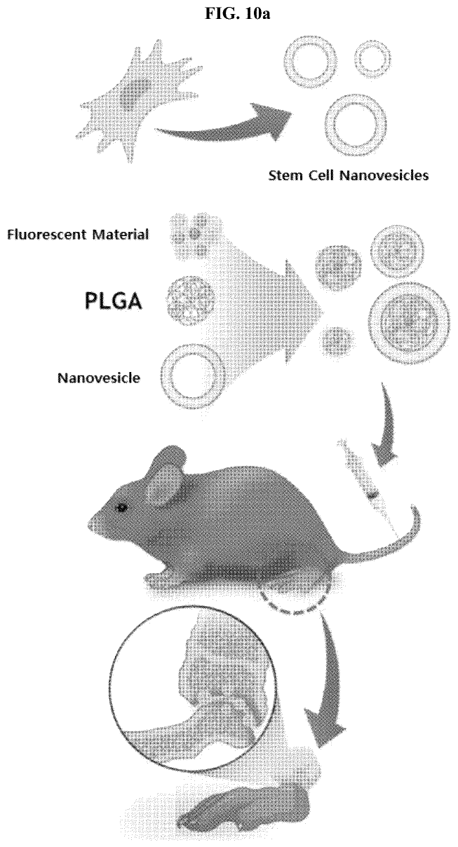

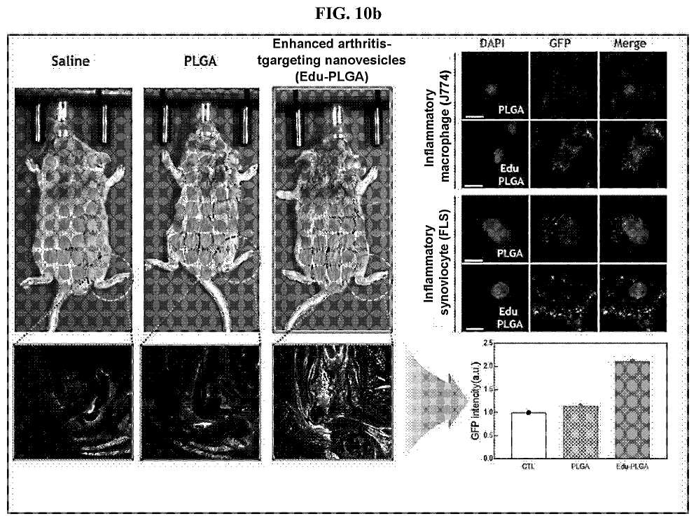

Furthermore, the present inventors investigated whether the above enhancement of tumor cell targeting would be the same for other lesions, such as inflamed tissue. Specifically, the present inventors prepared ectosome-PLGA nanoparticle complexes by isolating ectosomes from adipose-derived stem cells educated with fibroblast-like synoviocytes (FLSs) isolated from inflamed tissue, and by coating them on PLGA nanoparticles as described above. Subsequently, collagen-induced arthritis model animals were administrated with the ectosome-PLGA nanoparticle complexes, and it was found that the ectosome-PLGA nanoparticle complexes aggregated specifically in inflamed tissue (see FIG. 10b).

These results suggest that the stem cell-derived ectosome-biodegradable polymer nanoparticle complexes educated according to one embodiment of the present invention may be useful as drug delivery carriers of active drugs against the same kind of lesions as the educated lesion-derived cells, by increasing their targeting ability against those lesions.

The present invention will now be described in more detail with reference to the following examples. However, the invention is not limited to the embodiments disclosed herein, but may be embodied in many different forms, and the following embodiments are provided to make the disclosure of the invention complete and to give those of ordinary skill in the art a complete idea of the scope of the invention.

### Example 1: Isolation of Ectosomes (Nanovesicles) from Adipose-Derived Mesenchymal Stem Cells (AD-MSCs) that have been Educated with Cancer Cells to Enhance their Targeting Ability

Ectosomes (nanovesicles) were isolated for use in the preparation of polymers (PLGA) coated with ectosomes (nanovesicles) derived from adipose-derived mesenchymal stem cell (ADMSC) that have enhanced targeting abilities due to the education in cancer cell media according to one embodiment of the present invention.

### 1-1: How to Educate Stem Cells

To enhance the targeting ability to cancer cells, the present inventors seeded adipose-derived mesenchymal stem cells (AD-MSCs), pancreatic cancer cells (PANC-1), and brain tumor cells (U87MG) at 5×10⁴ per well in 100πL culture plates and incubated them for 48 hours at 37° C. The culture fluid (medium) was collected from the plates with pancreatic cancer cells or brain tumor cells in culture, and 5 ml was dispensed into the stem cells in culture, for further incubation for 24 hours (education).

### 1-2: Isolation of Ectosomes from Educated Stem Cells

5 μl of cytochalasin B (10 μg/l) was added to a plate containing adipose-derived mesenchymal stem cells (AD-MSCs) educated with cancer cell culture medium. Cells were detached and vortexed for 3 minutes. The supernatant was collected by centrifugation for 10 min at 1000 rpm using a centrifuge, and the supernatant was removed by centrifugation for 15 min at 4,000 rpm to obtain an ectosome pellet, which was resuspended in 10 μl of DW and quantified using a nanodrop.

### Example 2: Ectosome-PLGA Coating

### 2-1: Preparation of PLGA Nanoparticles

Polylactate-co-glycolate (PLGA) is a typical biodegradable polymer. To proceed with PLGA synthesis, after running the tip sonicator (30 hz), 1 ml of PLGA aerosol ([CAS #: 26780-50-7], 50:50 Carboxylated End Group (nominal), Lactel, Part #B6013-2P) were dissolved at 5 mg/ml in the solvent acetone to prepare a PLGA solution. Subsequently, 1 ml of the PLGA solution was added to 3 ml of 1% PVA. The solvent, acetone, was removed by concentrating under vacuum for 10 minutes using a rotary evaporator. The supernatant was then removed by centrifugation at 4° C. and 17,000 xg for 10 minutes to remove the remaining PLGA that did not form nanoparticles. The amount of PLGA nanoparticles produced was then quantified using a UV-VIS spectrophotometer.

### 2-2: Preparation of Ectosome-Coated PLGA Nanoparticles

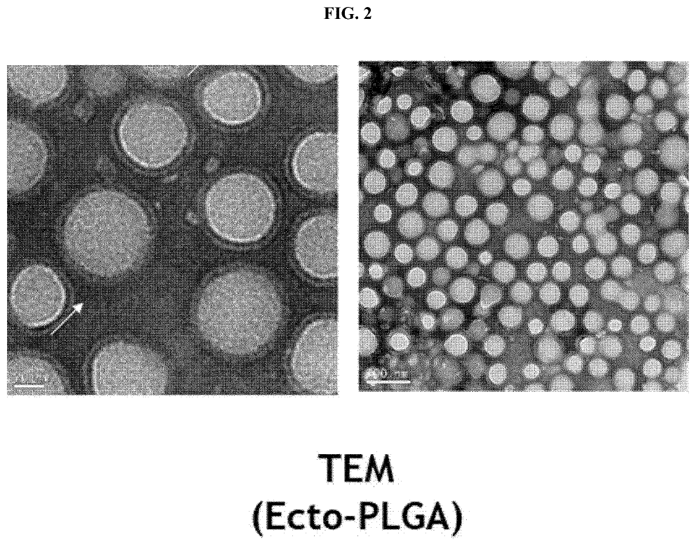

The ectosomes extracted in Example 1 above and the PLGA nanoparticles prepared in Example 2-1 above were mixed at 1:10 (mass ratio) and sonicated in a bath sonicator for 5 minutes. Transmission electron microscopy (TEM) was used to confirm that the ectosomes were properly attached to the PLGA (FIG. 2).

### Example 3: Preparation of DID, DIO-Labeled Ectosome-PLGA

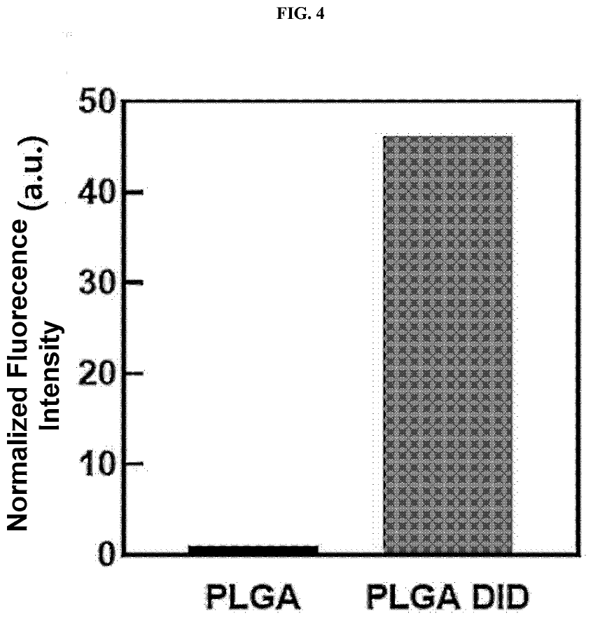

For the preparation of ectosome-PLGA complexes embedded with the fluorescent dye DiD or DIO, the present inventors operated a tip sonicator at 30 hz, with a mixture of 3 ml of 1% PVA and either DID (1,1′-Dioctadecyl-3,3,3′,3′-Tetramethylindodicarbocyanine, 4-chloro-benzenesulfonate salt, D7757, Thermo Fisher Scientific, USA) or DIO (3,3′-Dioctadecyl-oxacarbocyanine Perchlorate, D275, Thermo Fisher Scientific, USA), where 1 ml of 5 mg/ml of PLGA dissolved in acetone was added thereafter. The solvent, acetone, was then removed using a rotary evaporator for 10 minutes under vacuum. Then, centrifugation was performed using a centrifuge at 4° C. and 17,000 xg for 10 minutes to remove the supernatant and thereby remove the residual PLGA that did not form nanoparticles. Meanwhile, the resulting PLGA nanoparticles were quantified using a UV-VIS spectrophotometer. Then, the ectosomes isolated in Example 1 above and the DID or DIO prepared above were mixed at 1:10 (mass ratio) and sonicated in a bath sonicator for 5 minutes. After that, fluorescence was checked using a Victor 1420 Multilabel Counter (PerkinElmer, Waltham, MA, USA) at 646 nm excitation wavelength and 661 nm emission wavelength. As a result, as shown in FIG. 4, the DID- or DIO-embedded PLGA showed proper fluorescence.

### Experimental Example 1: Physicochemical Properties

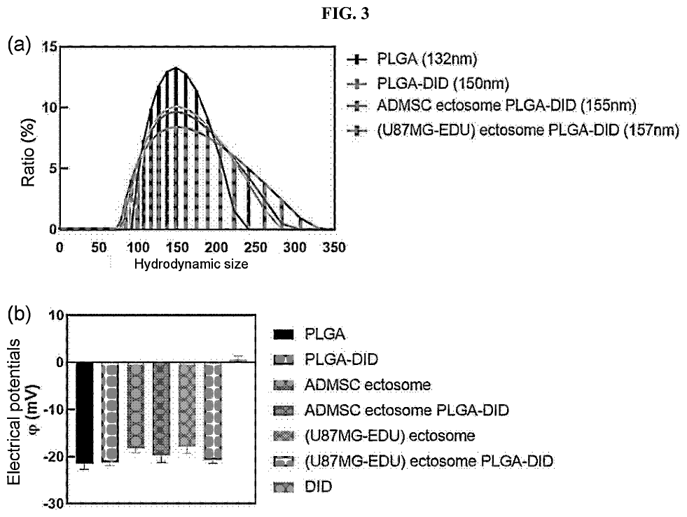

The size distribution and electrical potential of the ectosome-PLGAs were analyzed using a nanoparticle size analyzer (Anton Paar LiteSizer 500). As a result, as shown in FIG. 3, ectosome-PLGA nanoparticle complexes according to one embodiment of the present invention exhibited similar particle size (FIG. 3a) and potential to bare PLGA nanoparticles (FIG. 3b). Interestingly, the particle sizes of both PLGA nanoparticles and ectosome-coated PLGA nanoparticles were very similar to that of the ectosomes.

### Experimental Example 2: Analysis of the Uptake Capacity of Ectosome-PLGA Nanoparticle Complexes into Tumor Cells

The present inventors investigated the capacity of ectosome-PLGA nanoparticle complexes according to one embodiment of the present invention for their uptake into tumor cells. Specifically, the present inventors seeded brain tumor cells U87MG and pancreatic cancer cells PANC-1 onto poly-d-lysine-coated coverslips (5×10⁴ cells) and incubated them overnight. Then, 0.5 μg/mL of PLGA nanoparticles (PLGA) labeled with DIO which is a fluorophore in the GFP wavelength band, ectosome-PLGA nanoparticle complexes isolated from uneducated adipose-derived stem cells labeled with DIO (ADMSC-PLGA), and ectosome-PLGA nanoparticle complexes isolated from adipose-derived stem cells educated according to one embodiment of the present invention labeled with DIO were added to the cells in culture and incubated at 37° C. for 6 hours. After 6 hours, all samples were fixed with 4% paraformaldehyde in medium at 4° C. overnight, and the DIO fluorescence image of the tumor cells was obtained using an LSM700 confocal microscope (Carl Zeiss). As a result, as shown in FIG. 5, no fluorescence was detected inside the tumor cells treated with bare PLGA nanoparticles, but fluorescence was observed inside the tumor cells treated with the uneducated stem cell-derived ectosome-PLGA nanoparticle complex. However, the intensity of the fluorescence was lower compared to that of the stem cell-derived ectosome-PLGA nanoparticle complex educated according to one embodiment of the present invention.

### Experimental Example 3: Analysis of the Tumor Tissue Targeting Ability of Ectosome-PLGA in a Pancreatic Cancer Model

According to one embodiment of the present invention, the targeting ability of ectosome-PLGA was analyzed in an in vivo animal model.

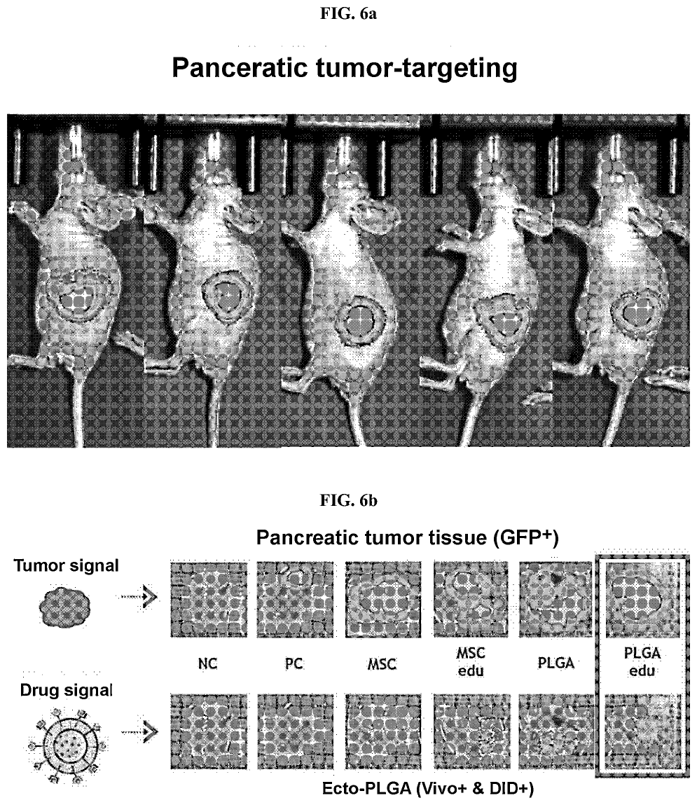

Specifically, BALB/c nude mice were injected with pancreatic cancer cells genetically engineered to express a fluorescent protein and bioluminescent enzyme (luciferase) (PANC-1-luciferase-GFP) at 1×10⁶ through the tail vein to create pancreatic cancer models. After 4 weeks, the size of the pancreatic cancer was checked using a biofluorescence imaging device (IVIS imaging system) to confirm the formation of pancreatic cancer, and then ectosome-PLGAs isolated from AD-MSCs educated with pancreatic cancer cells (1×10⁶ cells, 5 mg/kg), free AD-MSCs (1×10⁶ cells), educated AD-MSCs (1×10⁶ cells), and PLGA (5 mg/kg) were injected intravenously through the tail vein of the above pancreatic cancer model mice once a day for a total of 3 days. On day 4, whole body bioimaging was performed on the experimental animals. After sacrificing the experimental animals, the tumor-bearing pancreases were harvested and ex vivo imaging was performed to analyze the targeting ability of ectosome-PLGA to the tumor tissue (FIGS. 6a and 6b).

As a result, as shown in FIGS. 6a and 6b, the ectosome-PLGAs educated by treating cancer cell culture media showed enhanced targeting ability to tumor tissue, suggesting that stem cells educated according to one embodiment of the present invention can be used very efficiently for cancer cell-specific drug delivery, particularly for drug delivery against pancreatic cancer, which is a representative incurable malignancy.

### Example 4: Preparation of Doxorubicin-Loaded Ectosome-PLGA

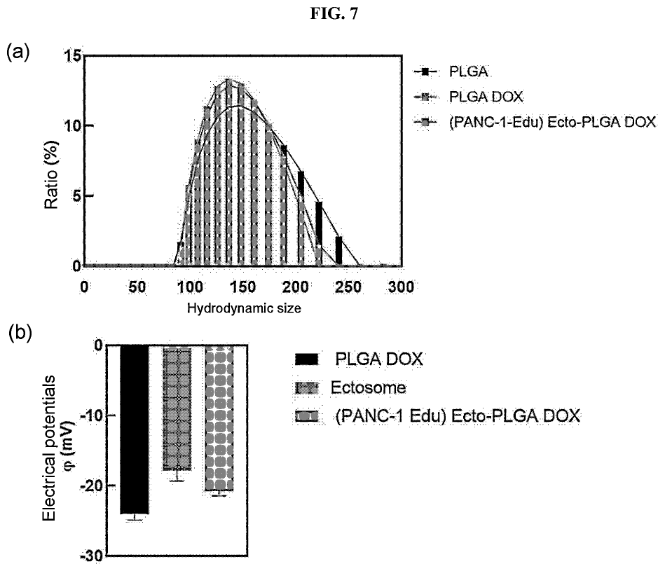

For the preparation of doxorubicin-loaded ectosome-PLGA, the present inventors prepared a PLGA solution by dissolving PLGA copolymer ([CAS #: 26780-50-7], 50:50 Carboxylated End Group (nominal), Lactel, Part #B6013-2P) at 5 mg/ml in acetone, and added 1 ml of it to 3 ml of 1% PVA solution mixed with 600 μl of doxorubicin (1 mg/ml) under operation of a tip sonicator at 30 hz. Then, the solvent, acetone, was removed by concentrating under vacuum for 10 min using a rotary evaporator. The supernatant was then removed by centrifugation at 4° C., 17,000 xg for 10 min using a centrifuge to remove the remaining PLGA that did not form nanoparticles. Then, a UV-VIS spectrophotometer was used to quantify the amount of PLGA nanoparticles produced, and the particle sizes and electrical potentials of the resulting complexes were analyzed in the same manner as in Example 1 above. As a result, as shown in FIG. 7, the doxorubicin-loaded ectosome-PLGA nanoparticle complexes derived from educated stem cells also exhibited similar particle size distribution (FIG. 7a) and electrical potential (FIG. 7b) as the PLGA nanoparticles or the doxorubicin-loaded PLGA nanoparticles.

### Experimental Example 4: Analysis of the Tumor Tissue Targeting Ability of Ectosome-PLGA in a Brain Tumor Model

According to one embodiment of the present invention, the targeting ability of ectosome-PLGA was analyzed in an in vivo animal model.

Specifically, brain tumor cells (U87MG-luciferase-GFP) genetically engineered to express fluorescent protein and bioluminescent enzyme (luciferase) were formed in BALB/c nude mice by surgically injecting 3×10⁵ of the U87MG-luciferase-GFP brain tumor cells directly into the brain. After confirming the formation of brain tumors by checking the brain tumor sizes using a IVIS imaging system, ectosome-PLGAs extracted from educated AD-MSC (1×10⁶ cells, 5 mg/kg), free AD-MSCs (1×10⁶ cells), educated AD-MSCs (1×10⁶ cells), and PLGA (5 mg/kg) were injected intravenously through the tail vein of the above brain tumor model mice once a day for a total of 3 days. On day 4, whole body bioimaging was performed on the experimental animals, and the experimental animals were sacrificed for the harvest of tumor-bearing brain cancers. Ex vivo imaging was performed to analyze the targeting ability of ectosome-PLGA to tumor tissue.

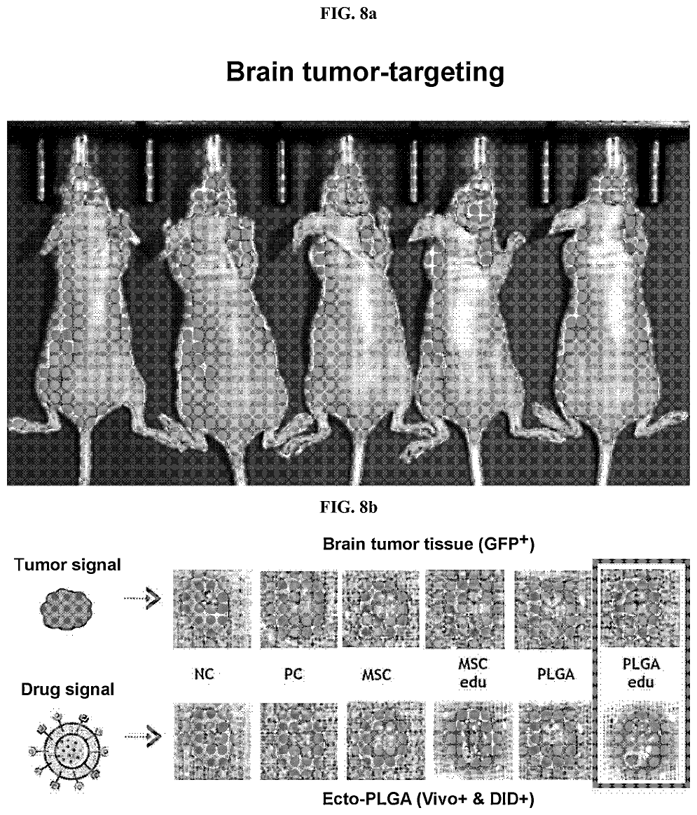

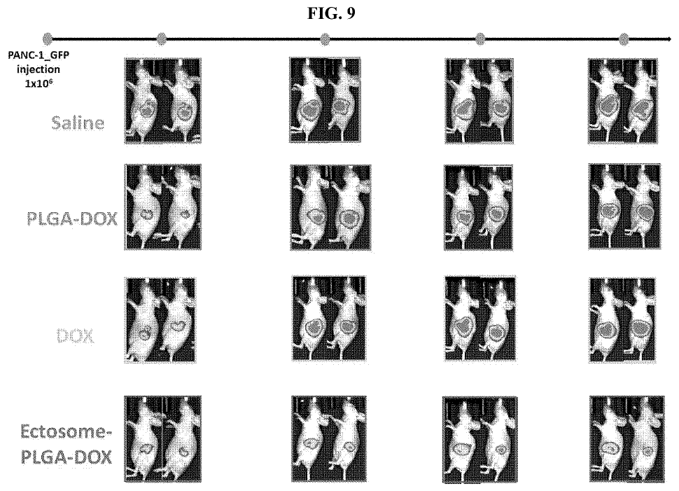

As a result, as shown in FIGS. 8a and 8b, it was found that the targeting ability to brain tumor tissue was enhanced for the ectosome-PLGA educated by treating the cancer cell culture medium. Also, the experimental results, achieved by intravenous injection into experimental animals of biodegradable polymer nanoparticle complexes comprising not stem cells but stem cell-derived ectosome components, demonstrate that the ectosome-polymer nanoparticle complexes according to one embodiment of the present invention cross the blood-brain barrier and accurately migrate to brain tumors. This provides a clue to solving an important technical challenge in the treatment of brain tumors, where the presence of the blood-brain barrier has traditionally limited anticancer drug therapy.

### Experimental Example 5: Analysis of the Inhibitory Activity of Ectosome-PLGA Against Pancreatic Cancer

According to one embodiment of the present invention, the antitumor effect of ectosome-PLGA nanoparticles was analyzed in an in vivo animal model.

Specifically, in vivo fluorescence imaging, especially the IVIS animal fluorescence imaging system, was used to determine the cancer cell killing efficacy of ectosome-PLGAs in a fluorescent/luminescent PANC-1-luciferase-GFP tumor mouse models. First, nude mice were injected with pancreatic cancer cells (PANC-1) expressing luciferase-GFP fluorescence, and 4 weeks later, ectosome-PLGAs extracted from educated AD-MSCs (5 mg/kg) and PLGA-DOX (5 mg/kg) were injected twice a week for 3 weeks for a total of 6 times, and luciferase was activated by luciferin 150 mg/kg IV injection 1 hour before the measurement. Then the changes of PANC-1 fluorescence were analyzed hourly using the fluorescence imaging equipment.

The results showed that the administration of the ectosome-PLGA effectively inhibited the generation of pancreatic cancer cells expressing luciferase-GFP fluorescence, as shown in FIG. 9.

### Example 5: Isolation of Ectosomes (Nanovesicles) from Adipose-Derived Mesenchymal Stem Cells (AD-MSCs) with a Targeting Ability Enhanced by Education with Inflammatory Cell Culture Medium

According to one embodiment of the present invention, ectosomes (nanovesicles) were isolated for use in the preparation of polymer (PLGA) coated with ectosomes (nanovesicles) derived from adipose-derived mesenchymal stem cell (ADMSC) which were educated with inflammatory cell culture medium to enhance their targeting ability to inflammatory sites.

### 5-1: Education of Stem Cells

To enhance the ability to target inflammatory cells, the inventors seeded adipose-derived mesenchymal stem cells (AD-MSCs), inflammatory synoviocytes (FLS) and inflammatory macrophages (J774) at 5×10⁴ per well in 100π culture plates and incubated them for 48 hours at 37° C. The culture fluid (medium) from the plates with inflammatory cells was collected and 5 ml was dispensed into the stem cells in culture, for further incubation for 24 hours (education).

### 5-2: Isolation of Ectosomes from Educated Stem Cells

5 μl of cytochalasin B (10 μg/μl) was added to a plate containing adipose-derived mesenchymal stem cells (AD-MSCs) cultured with inflammatory cell culture medium. Cells were detached and vortexed for 3 minutes. The supernatant was collected by centrifugation for 10 min at 1000 rpm using a centrifuge, and then removed by centrifugation for 15 min at 4,000 rpm to obtain an ectosome pellet. Subsequently, the pellet was resuspended in 10 μl of DW and quantified using a nanodrop.

### Example 6: Ectosome-PLGA Coating

The coating of PLGA nanoparticles and DIO- or DID-labeled PLGA nanoparticles on ectosomes isolated from adipose-derived stem cells educated with inflammatory cells as in Example 5 above was performed identically to the method described in Example 2.

### Experimental Example 6: Analysis on Ectosome-PLGA Uptake in FLSs and J774 Cells

The present inventors investigated the uptake capacity into inflammatory cells of ectosome-PLGA nanoparticle complexes according to one embodiment of the present invention. Specifically, the present inventors seeded fibroblast-like synoviocytes (FLSs) and 774 cells (5×10⁴ cells) onto poly-d-lysine-coated coverslips and incubated them overnight. Then, 0.5 μg/mL of PLGA nanoparticles embedding DIO which is a fluorophore in the GFP wavelength range, and DIO-embedded ectosome-PLGA nanoparticle complexes isolated from adipose-derived stem cells educated according to one embodiment of the present invention were added to the cells and incubated at 37° C. for 6 hours. After 6 hours, all samples were fixed with 4% paraformaldehyde in medium at 4° C. overnight, and the DIO fluorescence in the inflammatory cells was photographed using an LSM700 confocal microscope (Carl Zeiss). As a result, no fluorescence was detected inside the inflammatory cells treated with bare PLGA nanoparticles, as shown in the upper right corner of FIG. 10b, while fluorescence was detected upon treatment with ectosome-PLGA nanoparticle complexes derived from stem cells educated according to one embodiment of the present invention.

### Example 7: Preparation of Collagen-Induced Arthritis (CIA) Models

DBA/1 mice (male, 4-6 weeks old, 20-25 g body weight) were purchased from Orient Bio (Seoul, Korea) and were housed in a temperature and humidity-controlled, specific pathogen-free environment with a 12 h light/dark cycle (lights on at 6:30 am) for 7-14 days and acclimatized prior to experiments. All animal experiments were performed in accordance with the Guide for the Care and Use of Laboratory Animals of Gachon University. Arthritis was also induced by tail-based intradermal injection of 2 mg/mL CFA (Chondrex). After the first signs of inflammation were observed on day 27, animals with AIA-induced arthritis were randomized into six groups (n=6).

### Experimental Example 7: Analysis on the Ability of Ectosome-PLGA to Target Arthritis in Arthritis Models

Specifically, to evaluate the ability of ectosome-PLGA to target arthritis in DBA/1 mice as arthritis models, CIA-induced mice were prepared by intradermal injection of complete Freund's adjuvant (CFA) (FIG. 10a) (L. Bevaart, et al., Arthritis Rheum. 66-3362(8): 2192-2205, 2010). The targeting ability of ectosome-PLGA to arthritic cells was analyzed using an IVIS imaging system and histological images. Ectosome-PLGA-DID (1×10⁶ cells, 5 mg/kg) and PLGA-DID (5 mg/kg) extracted from educated AD-MSCs were injected intravenously through the tail vein of arthritis model mice once a day for a total of 3 days. On day 4, whole-body bioimaging was performed on the experimental animals to analyze the targeting ability of ectosome-PLGA to arthritic cells. The results showed that the ectosome-PLGA nanoparticle complexes derived from stem cells educated with inflammatory cells according to one embodiment of the present invention were selectively accumulated in inflamed tissue, as shown in FIG. 10b. Thus, the biodegradable nanoparticle complexes coated with ectosome membrane derived from stem cells educated with lesion-derived cells such as tumor or inflammation according to one embodiment of the present invention, have site-specific targeting capabilities derived from the cells used for the education. This site-specific education can be used to prepare site-specific drug delivery carriers using ectosomes derived from stem cells if the stem cells are educated with cells that are highly relevant to the pathological conditions present at the site of origin, such as neurodegenerative diseases or various metabolic diseases, in addition to tumors and inflammation.

The present invention has been described with reference to the above embodiments, but these are exemplary only, and those having ordinary skill in the art will understand that various modifications and other equally valid embodiments are possible. The true scope of technical protection of the invention should therefore be determined by the technical ideas of the appended claims of the patent.

1. A biodegradable polymer nanoparticle coated with a membrane of stem cell-derived ectosome.

2. The biodegradable polymer nanoparticle of claim 1, wherein the stem cell is an embryonic stem cell, a mesenchymal stem cell, or an induced pluripotent stem cell.

3. The biodegradable polymer nanoparticle of claim 2, wherein the mesenchymal stem cells is a bone marrow-derived stem cell, a cord blood-derived stem cell, an adipose-derived stem cell, a pulp-derived stem cell, or a peripheral blood-derived stem cell.

4. The biodegradable polymer nanoparticle of 1, wherein the stem cell is a stem cell educated with lesion-derived cells.

5. The biodegradable polymer nanoparticle of 4, wherein the educated stem cell is a stem cell that has been cultured in contact with a culture medium in which the lesion-derived cells are being cultured.

6. The biodegradable polymer nanoparticle of claim 1, wherein the biodegradable polymer is a natural biodegradable polymer or an artificial biodegradable polymer.

7. The biodegradable polymer nanoparticle of 6, wherein the natural biodegradable polymer is starch, chitin, cellulose, polyalginate, or collagen.

8. The biodegradable polymer nanoparticle of claim 6, wherein the artificial biodegradable polymer is PLGA {poly(lactic-co-glycolic) acid)}, PGA {poly(glycolic acid)}, PLA {poly(lactic acid)}, PCL {poly(caprolactone)}, or PHA (polyhydroxyalkanoate).

9. The biodegradable polymer nanoparticle of claim 1, wherein the nanoparticle has a size of 100 to 350 nm in diameter.

10. A drug delivery carrier comprising the nanoparticle of claims 1.

11. A pharmaceutical composition comprising the drug delivery carrier of claim 10 and an active drug loaded to the drug delivery carrier.

12. The pharmaceutical composition of claim 11, wherein the active drug is an anti-cancer agent or an anti-inflammatory agent.

13. A pharmaceutical composition for the treatment of cancer comprising a nanoparticle-drug complex comprising the biodegradable polymer nanoparticle of claim 1 and an anti-cancer agent loaded to the nanoparticle as an active ingredient.

14. The pharmaceutical composition of claim 13, wherein the stem cell is a stem cell educated with target cancer cells.

15. The pharmaceutical composition of claim 13, wherein the anticancer agent is loaded on the surface of or inside the nanoparticle by covalent or non-covalent bond or by inclusion within the nanoparticle having a core-shell structure.

16. A method of treating cancer comprising:
administering the pharmaceutical composition of claim 13 to a subject in need thereof.

17. A pharmaceutical composition for the treatment of inflammation comprising a nanoparticle-drug complex comprising the biodegradable polymer nanoparticle of claim 1 and an anti-inflammatory agent loaded to the nanoparticle as an active ingredient.

18. The pharmaceutical composition of claim 17, wherein the stem cell is a stem cell educated with inflammatory cells obtained from target inflammatory sites.

19. The pharmaceutical composition of claim 17, wherein the anti-inflammatory agent is loaded on the surface of or inside the nanoparticle by covalent or non-covalent bond or by inclusion within the nanoparticle having a core-shell structure.

20. A method of treating inflammation in a subject suffering from inflammation, comprising administering the pharmaceutical composition of claim 17.

21. A method of preparing an ectosome-biodegradable polymer nanoparticle complex with enhanced targeting ability to lesion site, comprising preparing an educated stem cell by educating the stem cell with lesion-derived cells; isolating ectosome from the educated stem cell; and producing ectosome membrane-coated biodegradable polymer nanoparticle by mixing the ectosome or ectosome membrane prepared by degradation of the ectosome with a biodegradable polymer nanoparticle.

22. The method according to claim 21, wherein the ectosome membrane is produced by mechanical extrusion or sonication of the ectosome.

23. The method according to claim 21, wherein the stem cell is an embryonic stem cell, a mesenchymal stem cell, or an induced pluripotent stem cell.

24. The method according to claim 21, wherein the mesenchymal stem cell may be a bone marrow-derived stem cell, a cord blood-derived stem cell, an adipose-derived stem cell, a pulp-derived stem cell, or a peripheral blood-derived stem cell.

25. The method according to claim 21, wherein the educating the stem cell is performed by culturing the stem cell in contact with a culture medium in which the lesion-derived cells are being cultured.

26. The method according to claim 21, wherein the biodegradable polymer is a natural biodegradable polymer or an artificial biodegradable polymer.

27. The method according to claim 21, wherein the natural biodegradable polymer is starch, chitin, cellulose, polyalginate, or collagen.

28. The method according to claim 21, wherein the artificial biodegradable polymer is PLGA {poly(lactic-co-glycolic) acid)}, PGA {poly(glycolic acid)}, PLA {poly(lactic acid)}, PCL {poly(caprolactone)}, or PHA (polyhydroxyalkanoate).

29. The method according to claim 21, wherein the nanoparticle has a size of 100 to 350 nm in diameter.
